(19) United States
(12) Patent Application Publication
(10) Pub. No.: US 2024/0252574 A1
(43) Pub. Date: Aug. 1, 2024

(54) COMPOSITION CONTAINING NATURAL PRODUCT-DERIVED ESSENTIAL OIL EXTRACT AS ACTIVE INGREDIENT FOR IMPROVING RESPIRATORY DISEASES

(71) Applicant: BOINBIO-CONVERGENCE CO., LTD., Seoul (KR)

(72) Inventors: Woong Mo YANG, Hanam-si (KR); Yew-Min Tzeng, Seoul (KR)

(73) Assignee: BOINBIO-CONVERGENCE CO., LTD., Seoul (KR)

(21) Appl. No.: 18/565,815

(22) PCT Filed: Apr. 28, 2022
(86) PCT No.: PCT/KR2022/006126

§ 371 Date: Nov. 30, 2023

(30) Foreign Application Priority Data
May 31, 2021 (KR) 10-2021-0069840

(51) Int. Cl. A61K 36/534; A61K 9/00; A61K 36/15; A61K 36/268; A61P 11/00; A61P 29/00

(52) U.S. Cl. CPC A61K 36/534; A61K 9/0056; A61K 9/0073; A61K 36/15; A61K 36/268; A61P 11/00; A61P 29/00; A61K 2236/331

(57) ABSTRACT

The present invention relates to a composition for treating respiratory diseases due to particulate matter, wherein the composition contains, as active ingredients, essential oil extracts of mint, Asarum sieboldi, and fir leaves. A composite obtained by mixing essential oil extracts of mint, Asarum sieboldi, and fir leaves according to the present invention exhibits the effect of relieving the symptoms of chronic respiratory diseases caused by ovalbumin and particulate matter in an animal model, and thus can be effectively used to directly treat respiratory diseases due to particulate matter.

### TECHNICAL FIELD

The present invention relates to a composition for improving respiratory diseases comprising natural product-derived essential oil extracts as active ingredients, and more particularly, to a composition for preventing, relieving, or treating respiratory diseases due to particulate matter, comprising essential oil extracts of mint, Asarum sieboldi, and fir leaves as active ingredients.

### BACKGROUND ART

Recently, as the concentration and duration of particulate matter (PM) increases, interest in effects of particulate matter on the human body is increasing (Kim et al., 2017). Due to desertification of the Asian continent, including China, etc., according to climate change, an influence of yellow dust originating from the desert areas of Mongolia and China and the Huanghua River basin is increasing the occurrence of yellow dust in Korea, and recently, the rapid industrialization of Northeast Asian countries has been reported as the main cause of particulate matter.

The particulate matter includes various components, including carbon components such as soot and biological organic carbon, ionic components such as chlorine, nitric acid, ammonium, sodium, and calcium, metal components such as lead, arsenic, and mercury, polycyclic aromatic hydrocarbons such as benzopyrene, etc. In addition, primary particles emitted from automobile exhaust gases, quarries, and construction sites, and secondary particles such as sulfates, nitrates, sulfur dioxide, nitrogen oxides, ammonia, and volatile organic compounds generated through chemical reactions, affect the occurrence of particulate matter.

Ultra-particulate matter (PM_{2.5}) refers to particulate matter with an aerodynamic diameter of 2.5 μm or less, and mainly includes secondary air pollutants (NO₃—, SO₄—, NH₄—, polyacromatic hydrocarbon (PAH), quinone, etc.) produced by reacting in the atmosphere with primary pollutants emitted directly from air pollution sources (Kim et al., 2017). According to the World Health Organization (2013), it was reported that long-term exposure to PM₁₀ (particulate matter with an aerodynamic diameter of 10 μm or less) increased respiratory tract-related diseases and mortality, but PM_{2.5} acted as a stronger risk factor therethan.

General dust is caught in the nose or throat and does not affect the trachea, but dust with a diameter of 5 to 10 μg/m3 or less can be absorbed into the body through the nasal mucosa, dust with a diameter of 2 to 5 μg/m3 or less passes through the trachea (respiratory tract) and is deposited even in the upper trachea, bronchi, small trachea and alveoli to affect the respiratory system and cause allergic rhinitis, bronchitis, asthma, etc., and dust with a diameter of 0.1 to 1 μg/m3 causes the alveolar damage. When the particulate matter is inhaled into the human body, the particulate matter may be deposited in tissues through various mechanisms such as collision, gravitational sedimentation, diffusion, and electrostatic adsorption, and some thereof may circulate throughout the body along the blood.

In particular, it has been reported that particulate matter deposited in the body induced oxidative stress and inflammatory responses and caused acute exacerbation, etc. of respiratory and circulatory diseases (Myung, 2016). In addition, when the disease progresses to chronic inflammation, the particulate matter may cause chronic obstructive pulmonary disease (COPD), which causes breathing difficulties due to decreased lung function. The particulate matter causes not only respiratory diseases, but also allergic conjunctivitis, keratitis, cardiovascular diseases, cranial nerve diseases, etc., and it is known that these effects on the human body are caused by inflammatory reactions due to secretion of cytokines, chemokines, etc., an increase in the number of white blood cells, and production of active oxygen. As a result, research on materials capable of inhibiting these effects is required. Accordingly, research is required on natural food materials capable of suppressing oxidative stress and inflammatory responses in the human body that may be induced by such particulate matter.

In order to solve the problems, the present inventors studied natural materials capable of suppressing diseases caused by particulate matter, and as a result, confirmed that a composite mixed with extracts extracted from mint, Asarum sieboldi, and fir leaves showed efficacy in relieving symptoms of respiratory diseases caused by particulate matter in an animal model, and then completed the present invention.

### DISCLOSURE

### Technical Problem

An object of the present invention is to provide a pharmaceutical composition for preventing or treating respiratory diseases.

Another object of the present invention is to provide a health functional food composition and a food composition for preventing or relieving respiratory diseases.

Yet another object of the present invention is to provide an inhaled preparation for preventing, relieving, or treating respiratory diseases.

Yet another object of the present invention is to provide a method for preventing or treating respiratory diseases using the pharmaceutical composition.

### Technical Solution

In order to achieve the object, an aspect of the present invention provides a pharmaceutical composition for preventing or treating respiratory diseases including extracts of mint, Asarum sieboldi, and fir leaves as active ingredients.

In an embodiment of the present invention, the extracts of mint, Asarum sieboldi, and fir leaves may be essential oil extracts of mint, Asarum sieboldi, and fir leaves.

In an embodiment of the present invention, the extracts of mint, Asarum sieboldi, and fir leaves may be a composite in which the extracts of mint, Asarum sieboldi, and fir leaves are mixed in a volume ratio of 4:1 to 3:2 to 4, preferably in a volume ratio of 4:1.5 to 2.5:2.5 to 3.5.

In an embodiment of the present invention, the respiratory diseases may be respiratory diseases due to particulate matter.

In an embodiment of the present invention, the composition may be administered by inhalation or intranasal administration.

Another aspect of the present invention provides a health functional food composition and a food composition for preventing or relieving respiratory diseases including extracts of mint, Asarum sieboldi, and fir leaves as active ingredients.

Yet another aspect of the present invention provides an inhaled preparation for preventing, relieving or treating respiratory diseases including extracts of mint, Asarum sieboldi, and fir leaves as active ingredients.

Yet another aspect of the present invention provides a method for preventing or treating respiratory diseases including administering the pharmaceutical composition according to the present invention to a patient.

### Advantageous Effects

According to the present invention, it was confirmed through animal experiments that a composite obtained by mixing essential oil extracts of mint, Asarum sieboldi, and fir leaves exhibits the effect of reducing increased epithelial thickness and collagen accumulation in lung tissue caused by ovalbumin and particulate matter, reducing the expression of immunoglobulin A in trachea tissue and the concentration of immunoglobulins E and G in lung tissue serum, and reducing the expression of inflammatory cytokines. Therefore, the composite of the present invention can be effectively used for directly preventing, relieving, and treating respiratory diseases due to particulate matter.

### DESCRIPTION OF DRAWINGS

FIG. 1 is a schematic diagram illustrating an experimental schedule using an animal model inducing chronic respiratory diseases of the present invention.

FIG. 2 illustrates histological changes in lung tissue according to treatment with a composite of essential oil extracts of mint, Asarum sieboldi, and fir leaves of the present invention through hematoxylin & eosin staining. A yellow bar represents the thickness of the epithelium, and the results were expressed as mean±standard error of mean (###p<0.001 vs. NOR group: *** p<0.001 vs. OVA+PM10 group).

FIG. 3 illustrates the degree of collagen deposition in lung tissue according to treatment with the composite of the present invention through Masson trichrome staining. A blue region represents the collagen accumulation, and the results were expressed as mean±standard error of mean (###p<0.001 vs. NOR group: *** p<0.001 vs. OVA+PM10 group).

FIG. 4 illustrates the expression of IgA in trachea tissue according to treatment with the composite of the present invention through immunohistochemical staining. A red region means the expression of IgA.

FIG. 5 illustrates the levels of serum immunoglobulins E and G (IgE and IgG) in lung tissue according to treatment with the composite of the present invention through ELISA. Results were represented as mean±standard error of mean (##p<0.05 and ###p<0.001 vs. NOR group: * p<0.05 and *** p<0.001 vs. OVA+PM10 group).

FIG. 6A illustrates the expression of pro-inflammatory cytokine of TNF-α in lung tissue according to treatment with the composite of the present invention. Results were represented as mean±standard error of mean (##p<0.05 and ###p<0.001 vs. NOR group: * p<0.05, ** p<0.01 and *** p<0.001 vs. OVA+PM10 group).

FIG. 6B illustrates the expression of pro-inflammatory cytokine of IL-6 in lung tissue according to treatment with the composite of the present invention. Results were represented as mean±standard error of mean (##p<0.05 and ###p<0.001 vs. NOR group: * p<0.05, ** p<0.01 and *** p<0.001 vs. OVA+PM10 group).

### BEST MODE OF THE INVENTION

Hereinafter, embodiments of the present invention will be described in detail with reference to the accompanying drawings. In the following description, detailed descriptions of techniques well-known to those skilled in the art may be omitted. Further, in describing the present invention, the detailed description of associated known functions or constitutions will be omitted if it is determined that they unnecessarily make the gist of the present invention unclear. Terminologies used herein are terminologies used to properly express preferred embodiments of the present invention, which may vary according to a user, an operator's intention, or customs in the art to which the present invention pertains.

Accordingly, definitions of the terminologies need to be described based on contents throughout this specification. Throughout the specification, unless explicitly described to the contrary, when a certain part “comprises” a certain component, it will be understood to imply the inclusion of stated elements but not the exclusion of any other elements.

The present invention provides a pharmaceutical composition for preventing or treating respiratory diseases including extracts of mint, Asarum sieboldi, and fir leaves as active ingredients.

The mint (Menthae Herba) of the present invention refers to the top part of Mentha arvensis or Mentha piperita. Mentha arvensis Linne var. piperascens is a perennial herb of the Dicotyledoneae, Tubiflorales, Labiatae, and the plant itself is aromatic, erect, and branched at the top. The mint has anti-inflammatory and analgesic effects and is known to be effective in various symptoms accompanied by skin diseases such as hives and rashes and fever, and has been also used for muscle pain and overwork.

The Asarum sieboldi (Asiasari Radix et Rhizoma) of the present invention means roots and rhizomes of Asiasarum heterotropoides F. Maekawa var. mandshuricum F. Maekawa or Asiasarum sieboldii Miquel var. seoulense Nakai of Aristolochiaceae. The Asarum sieboldi is known to have antibacterial, analgesic, and sedative effects, and recently, research has been actively conducted on anti-inflammatory inhibition and antioxidant response, regulation of vascular smooth muscle, and anti-allergic effects of the Asarum sieboldi extract.

The fir leaves of the present invention refer to the leaves of fir (Abies holophylla), also called manchurian fir, which is an evergreen tree of the gymnosperm Coniferophyta, Coniferales, Pinaceae. The leaves and branches of the fir tree have been collected and used medicinally for uterine bleeding, gastrointestinal disease, gum disease, diarrhea, and the like, and it is known that the essential oil of fir leaves not only has a relieving effect when used on skin with skin courap such as itching, swelling, wounds, boils, and lumps, but also is excellent on alleviation of skin problems such as dry skin and rashes, psoriasis, vitiligo, and metabolism promotion of the skin.

In the present invention, the ‘extract’ refers to a preparation obtained by squeezing an extractive with an appropriate leachate or extraction solvent and concentrating the extractive by evaporating the leachate or extraction solvent, and is not limited thereto, but may be an extract obtained through extraction, a diluted or concentrate of the extract, a dried product obtained by drying the extract, and a crude or purified product thereof.

In the present invention, the extracts of mint, Asarum sieboldi, and fir leaves may be essential oil extracts of mint, Asarum sieboldi, and fir leaves extracted from the mint, Asarum sieboldi, and fir leaves, respectively.

In the present invention, the ‘essential oil’ is a plant essence containing biochemical components that are produced from special cells on the surface or tissue of plants, defend themselves from an external environment, and are self-produced for reproduction and survival. In addition, the ‘essential oil’ means a material that is extracted from the flowers, stems, fruits, roots, resin, and the like of aromatic medicinal plants and extracted by cold pressing, hydrodistillation, solvent extraction, supercritical carbon dioxide extraction, or the like depending on the characteristics of the plant. In general, the essential oil is pure natural vegetable oil with a unique plant aroma, and is a fat-soluble liquid, but is known to be non-sticky, light, and mostly colorless or pale yellow.

The extracts of mint, Asarum sieboldi, and fir leaves of the present invention may be obtained from the mint, Asarum sieboldi, and fir leaves through washing, drying, cutting and grinding, extracting and concentrating, and ripening steps, respectively, but are not limited thereto. The extracts of mint, Asarum sieboldi, and fir leaves may be extracted from mint, Asarum sieboldi, and fir leaves by conventional methods, for example, water extraction, water+alcohol extraction, alcohol extraction, hydrodistillation extraction, etc., respectively. It is obvious to those skilled in the art that the components and compositions contained in the extracts are different depending on the adopted extraction method. In the present invention, the preferable extracts of mint, Asarum sieboldi, and fir leaves may be a composite (Formula) obtained by mixing essential oil extracts of mint, Asarum sieboldi, and fir leaves obtained from the mint, Asarum sieboldi, and fir leaves through a hydrodistillation extraction method, respectively.

The hydrodistillation extraction method is known in the art, and specific examples are as described in Examples herein. The ratio of mint, Asarum sieboldi and fir leaves to purified water is 1:1 to 1:15, preferably 1:6 to 1:10, and more preferably 1:8 (by weight) to perform hydrodistillation extraction. The mint, Asarum sieboldi and fir leaves and purified water are each distilled in a distillation tank at 70° C. to 500° C., preferably 80° C. to 150° C., and more preferably 90° ° C. to 110° C. once or multiple times for about 3 hours to several days, preferably 4 to 10 hours, and more preferably 5 to 7 hours. The condensate obtained by cooling and condensing the distillate obtained above may be obtained and used. In addition, the obtained condensate may be further concentrated and filtered using a conventional method in the art to obtain higher-purity essential oil extracts of mint, Asarum sieboldi and fir leaves.

In the present invention, the extracts of mint, Asarum sieboldi, and fir leaves preferably refer to a composite produced by mixing single extracts extracted from mint, Asarum sieboldi, and fir leaves, respectively, but are not limited thereto, and may be a composite produced by mixing mint, Asarum sieboldi, and fir leaves and then extracting the mixture.

In the present invention, the extracts of mint, Asarum sieboldi, and fir leaves may be a composite obtained by mixing the mint extract, the Asarum sieboldi extract, and the fir leaves extract (that is, essential oil extracts of mint, Asarum sieboldi, and fir leaves) in a volume ratio of 4:1 to 3:2 to 4, preferably a composite obtained by mixing the mint extract, the Asarum sieboldi extract, and the fir leaves extract in a volume ratio of 4.0:1.5 to 2.5:2.5 to 3.5. When the extracts are mixed in a volume ratio within the range, the composite may exhibit a synergistic effect in the prevention or treatment of respiratory diseases and a significant effect, and when the volume ratio is out of the range, the effect may be reduced compared to an extract alone or a mixture of only some of the extracts or toxicity may occur.

In the present invention, the “respiratory diseases” are respiratory diseases due to inflammatory responses, hypersensitive immune responses (allergic responses), or both mechanisms, preferably respiratory diseases due to particulate matter.

The respiratory diseases due to particulate matter are respiratory diseases involved with inflammatory responses, hypersensitive immune responses or both mechanisms due to particulate matter, and may be any one selected from the group consisting of asthma, pneumonia, chronic obstructive pulmonary disease, rhinitis, bronchiectasis, acute and chronic bronchitis, bronchiolitis, pharyngitis, tonsillitis, laryngitis, idiopathic pulmonary fibrosis, cystic fibrosis, emphysema, pneumoconiosis, tuberculosis, sequelae of pulmonary tuberculosis, pulmonary fibrosis, lung cancer, lower respiratory infection, sinusitis, acute upper respiratory infection, and allergic lung disease, but are not limited thereto.

In the present invention, the composition may have an effect of reducing increased epithelial thickness and collagen accumulation in lung tissue.

In the present invention, the composition may have an effect of reducing the expression or concentration of one or more of immunoglobulins A, E, and G in lung tissue.

In the present invention, the composition may have an effect of reducing the expression of inflammatory cytokines in lung tissue.

In an embodiment of the present invention, it was confirmed that the composite obtained by mixing the essential oil extracts of mint, Asarum sieboldi, and fir leaves in a volume ratio of 4:2:3 had an effect of reducing increased epithelial thickness and collagen accumulation in lung tissue in a mouse model in which chronic respiratory diseases were induced by ovalbumin and particulate matter. It was confirmed that the composite reduced the expression level of immunoglobulin A (IgA) in trachea (respiratory tract) tissue and the concentration of immunoglobulins E and G (IgE and IgG) in lung tissue serum, and inhibited the expression of inflammatory cytokines TNF-α and IL-6 in lung tissue, thereby preventing, treating or relieving respiratory diseases (see Examples 1 to 5).

In the present invention, the ‘preventing’ refers to all actions that inhibit respiratory diseases or delay the progression of respiratory diseases by administering the composition of the present invention.

In the present invention, the ‘relieving’ refers to all actions that improve or beneficially change symptoms of respiratory diseases by administering the composition of the present invention.

In the present invention, unless otherwise stated, the ‘treating’ means reversing, alleviating, inhibiting the progression of, or preventing the disease or disorder to which the term is applied, or one or more symptoms of the disease or disorder, and as used herein, the term ‘treatment’ refers to any treating action when ‘treating’ is defined as above.

The term ‘administration’ used herein means providing the composition of the present invention in a predetermined pharmaceutically effective dose to a subject by any suitable method.

As used herein, the ‘pharmaceutically effective dose’ refers to an amount enough to treat the disease at a reasonable benefit/risk ratio applicable to medical treatment. The pharmaceutically effective dose may be determined according to factors including the type and severity of a disease of a subject, the activity of a drug, the sensitivity to a drug, a time of administration, a route of administration, an excretion rate, duration of treatment, and simultaneously used drugs, and other factors well-known in the medical field.

For administration, the pharmaceutical composition of the present invention may be preferably formulated as a pharmaceutical composition containing one or more pharmaceutically acceptable carriers in addition to the active ingredients. When formulated in the form of a liquid solution, the pharmaceutical composition is sterile and biocompatible, and saline, sterile water, Ringer's solution, buffered saline solution, albumin injection solution, dextrose solution, maltodextrin solution, glycerol, ethanol, or mixtures thereof may be used as a carrier, and other common additives such as antioxidants, buffers, and bacteriostatic agents may be added as needed. In addition, the pharmaceutical composition may be prepared in injectable formulations such as aqueous solutions, suspensions, and emulsions, pills, capsules, granules, or tablets by further adding a diluent, a dispersant, a surfactant, a binder, and a lubricant.

The pharmaceutical composition of the present invention may be administered to a subject through various routes. All methods of administration may be expected, but may be, for example, oral, intravenous, intramuscular, subcutaneous, and intraperitoneal injections, and preferably inhalation or intranasal administration. In an embodiment, the pharmaceutical composition for inhalation administration may typically be in the form of an aerosol or powder, and generally administered by using inhaler delivery devices, such as a dry powder inhaler (DPI), a metered-dose inhaler (MDI), a nebulizer inhaler, or a similar delivery device.

In an embodiment, the pharmaceutical composition may be administered by inhalation using a nebulizer inhaler. The nebulizer device typically generates a high-velocity airflow that nebulizes the pharmaceutical composition in mist and delivers the pharmaceutical composition to the respiratory tract of the patient. Accordingly, when formulated for use in the nebulizer inhaler, the composition may be dissolved in a suitable carrier to form a solution. Alternatively, the therapeutic agent may be micronized or nanomilled and combined with a suitable carrier to form a suspension. A representative pharmaceutical composition for use in the nebulizer inhaler may include a solution or suspension including about 0.0001 μL/mL to about 20 mL/mL of the extract of the present invention and excipients suitable for the nebulized formulation. The nebulizer device suitable for administering the therapeutic agent by inhalation is known in the art, and examples of the device are commercially available.

Further, the present invention provides a health functional food composition and a food composition for preventing or relieving respiratory diseases including extracts of mint, Asarum sieboldi, and fir leaves as active ingredients.

The “food” means natural products or processed products containing one or more nutrients, and preferably, means a condition that may be eaten directly through a certain amount of processing process, and as a general meaning, means including all foods, food additives, functional foods and beverages.

The food composition of the present invention may be formulated in the same manner as the pharmaceutical composition to be used as a functional food or added to various foods.

Foods to which the food composition may be added include, for example, various foods, beverages, gum, tea, vitamin complexes, functional foods, and the like. Additionally, the food includes special nutritional food (e.g., milk formulas, infant, baby food, etc.), processed meat products, fish products, tofu, jellied food, noodles (e.g., ramen, noodles, etc.), bread, health supplements, seasoned foods (e.g., soy sauce, soybean paste, red pepper paste, mixed soy sauce, etc.), sauces, confectionery (e.g., snacks), candies, chocolates, gums, ice creams, dairy products (e.g., fermented milk, cheese, etc.), other processed foods, kimchi, pickled foods (various types of kimchi, pickles, etc.), beverages (e.g., fruit drinks, vegetable drinks, soybean milk products, fermented drinks, etc.), and natural seasonings (e.g., ramen soup, etc.), but is not limited thereto. The foods, beverages or food additives may be prepared by general preparation methods.

As used herein, the term “functional food” or “health functional food” refers to a group of foods that have added value to the food to act and express the function of the food for a specific purpose by using physical, biochemical, and bioengineering techniques, or food that is designed and processed to sufficiently express body modulating functions for biological defense rhythm control, disease prevention and recovery, etc. of the food composition. Specifically, the “functional food” or “health functional food” may be a health functional food. The functional food may include food-acceptable food supplement additives, and may further include suitable carriers, excipients, and diluents which are commonly used in the preparation of functional foods.

The food composition of the present invention may contain various nutrients, vitamins, minerals (electrolytes), flavors, flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, natural carbohydrates, and the like, like conventional food compositions, in addition to the extracts as the active ingredients. In addition, the food composition of the present invention may contain pulp for preparing natural fruit juice, fruit juice beverages, and vegetable beverages.

Examples of the above-mentioned natural carbohydrates may include general sugars, such as monosaccharides, for example, glucose, fructose and the like: disaccharides, for example, maltose, sucrose and the like; and polysaccharides, for example, dextrin, cyclodextrin and the like, and sugar alcohols such as xylitol, sorbitol, erythritol, and the like. The above-mentioned flavoring agents may be advantageously used with natural flavoring agents (tauumatin), stevia extract (e.g., rebaudioside A, glycyrhizin, etc.), and synthetic flavoring agents (saccharin, aspartame, etc.).

The food composition of the present invention may be provided as a health functional food or health functional food composition, and may be manufactured and processed in the form of tablets, capsules, powders, granules, liquids, pills, beverages, etc.

The health functional food composition of the present invention may include conventional food additives, and the suitability as the food additives is determined by the specifications and standards for the corresponding item in accordance with the general rules of the Food Additive Codex, general test methods, and the like approved by the Food and Drug Administration, unless otherwise specified. The items disclosed in the “Food Additives Codex” may include, for example, chemical composites such as ketones, glycine, calcium citrate, nicotinic acid, cinnamic acid, and the like; natural additives such as desensitizing dye, licorice extract, crystal cellulose, Kaoliang color, guar gum, and the like: mixed formulations such as sodium L-glutamic acid formulations, noodle additive alkali agents, preservative formulations, tar color formulations, etc. For example, the health functional food in the form of tablets may formed by granulating a mixture obtained by mixing the active ingredients of the present invention with an excipient, a binder, a disintegrant, and other additives by the conventional method, and then compression-molding the mixture by adding a slip modifier and the like, or directly compressing the mixture. In addition, the health functional food in the form of tablets may also contain a flavor enhancer or the like as needed. In the health functional food in the form of capsules, hard capsules may be prepared by filling a mixture mixed with the active ingredients of the present invention and additives such as excipients into conventional hard capsules, and soft capsules may be prepared by filling a mixture mixed with the active ingredients of the present invention and additives such as excipients into capsule bases such as gelatin. The soft capsules may contain a plasticizer such as glycerin or sorbitol, a colorant, a preservative, and the like, if necessary. The health functional food in the form of pills may be prepared by molding a mixture obtained by mixing the active ingredients of the present invention with an excipient, a binder, a disintegrant, and the like by existing known methods, and may also be coated with white sugar or other coating agents or surface-coated with materials such as starch and talc, if necessary. The health functional food in the form of granules may be prepared by granulating a mixture obtained by mixing the active ingredients of the present invention with an excipient, a binder, a disintegrant, and the like by existing known methods and may contain a flavoring agent, a flavor enhancer, and the like, if necessary.

Furthermore, the present invention provides an inhaled preparation for preventing, relieving or treating respiratory diseases including extracts of mint, Asarum sieboldi, and fir leaves as active ingredients.

The inhaled preparation of the present invention may be selected from inhalation aerosol, inhalation powder, a liquid preparation for use in the nebulizer, or a preparation that may be converted to vapor. Preferably, the inhaled preparation may be selected from inhalation powder or a liquid preparation for use in the nebulizer, most preferably, a liquid preparation used in the nebulizer.

When the inhaled preparation of the present invention is used in the form of the inhalation powder, the inhaled preparation may further include one or more pharmaceutically acceptable additives. The “pharmaceutically acceptable additive” used herein may include one or more selected from surfactants, lubricants, and flavoring agents. For example, the pharmaceutically acceptable additive may be a surfactant such as phospholipids and poloxamer, magnesium stearate, micronized silica gel, a lubricant such as talcum powder, and a flavoring agent including a natural flavoring agent and a synthetic flavoring agent. The natural flavoring agent may include peppermint oil, orange peel oil, cinnamon oil, spearmint oil, mint water, compound orange spirit, etc., and the synthetic flavoring agent may include banana flavor, pineapple flavor, and orange flavor.

In an embodiment, the inhaled preparation of the present invention may further include injection water (physiological saline) together with the extracts of the present invention, and may be a liquid preparation used in a nebulizer, and the nebulizer may be a continuous nebulizer or a quantitative nebulizer.

When the inhaled preparation of the present invention is used as the liquid preparation used in the nebulizer, the inhaled preparation may further include one or more selected from an isotonic modifier, a pH modifier, a natural flavoring agent, and a synthetic flavoring agent. The isotonic modifier may be one or more selected from glucose, sodium chloride, potassium chloride, and mannitol, and the pH modifier may be one or more selected from sodium hydroxide, ammonium hydroxide, hydrochloric acid, sodium carbonate, sodium bicarbonate, dilute sulfuric acid, citric acid, sodium citrate, acetic acid, tartaric acid, sodium acetate and disodium hydrogen phosphate. The natural flavoring agent may be one or more selected from peppermint oil, orange peel oil, cinnamon oil, spearmint oil, mint water, and compound orange spirit, and the synthetic flavoring agent may be one or more selected from banana flavor, pineapple flavor, and orange flavor.

In an embodiment, the administration cycle of the inhaled preparation to the patient is selected from 3 times or less a day, 2 times or less a day, once or less a day, and once or less every other day, and preferably 2 times or less a day.

Furthermore, the present invention provides a method for preventing or treating respiratory diseases including administering the pharmaceutical composition for preventing or treating respiratory diseases to a patient.

The treatment method of the present invention includes administering the pharmaceutical composition to a subject in a therapeutically effective dose. It is preferred that a specific therapeutically effective dose for a specific subject is differently applied depending on various factors including the kind and degree of a response to be achieved, a specific composition including whether other agents are used in some cases, the age, body weight, general health conditions, sex, and diet of a subject, an administration time, an administration route, a secretion rate of the composition, a duration of treatment, and a drug used in combination or simultaneously with the specific composition, and similar factors well known in the medical field. Therefore, the effective dose of the composition suitable for the purpose of the present invention is preferably determined in consideration of the aforementioned matters.

The patient is applicable to any mammal, and the mammal includes not only humans and primates, but also livestock such as cattle, pigs, sheep, horses, dogs, and cats.

The present invention provides a method for preventing or treating respiratory diseases including administering the pharmaceutical composition for preventing or treating respiratory diseases including extracts of mint, Asarum sieboldi, and fir leaves in a pharmaceutically effective dose as active ingredients to a subject.

Hereinafter, the present invention will be described in more detail through Examples. However, these Examples are more specifically illustrative of the present invention, and the scope of the present invention is not limited to these Examples.

### MODES OF THE INVENTION

### Example 1. Preparation of Composite of Essential Oil Extracts of Mint, Asarum sieboldi and Fir Leaves

Essential oils were extracted from mint (Mentha arvensis, Mentha piperita), Asarum sieboldi (Asiasarum sieboldi), and fir (Abies holophylla) leaves by a hydrodistillation extraction method, respectively.

Specifically, 100 g of mint, 100 g of Asarum sieboldi, and 100 g of fir leaves were put into a 1 L round bottom flask equipped with a clevenger device, respectively, and then subjected to a hydrodistillation method for 6 hours at 100° C. to obtain essential oils of mint, Asarum sieboldi, and fir leaves, respectively. The obtained essential oils were 1.2 mL of mint (yield 1.2%), 1.2 mL of Asarum sieboldi (yield 1.2%), and 1 mL of fir leaves (1% yield). Each obtained essential oil was stored at 4ºC.

The essential oils of mint, Asarum sieboldi, and fir leaves were mixed in a volume ratio of 4:2:3 to prepare a composite (Formula).

### Preparation Example 1. Induction of Chronic Respiratory Disease and Animal Treatment

5-week-old female BALB/c mice were purchased and bred under a 12-hour light/dark cycle at a temperature of 22±2° C. and relative humidity of 55±10%. The mice were bred for one week by freely taking food and water, and used for experiments after a 7-day adaptation period.

The mice were randomly divided into 5 groups (n=7) and classified as follows: (1) NOR: a normal group, (2) OVA+PM10; a group in which mice exposed to ovalbumin (OVA) and particulate matter PM10 were treated with a vehicle as a negative control group: (3) DEX: a group in which mice exposed to OVA and particulate matter PM10 were treated with DEX as a positive control group: (4) Low: a group in which mice exposed to OVA and particulate matter PM10 were treated with 0.0009% (% v/v) of a low-concentration composite (Formula); and (5) High: a group in which mice exposed to OVA and particulate matter PM10 were treated with 0.09% (% v/v) of a high-concentration composite (Formula).

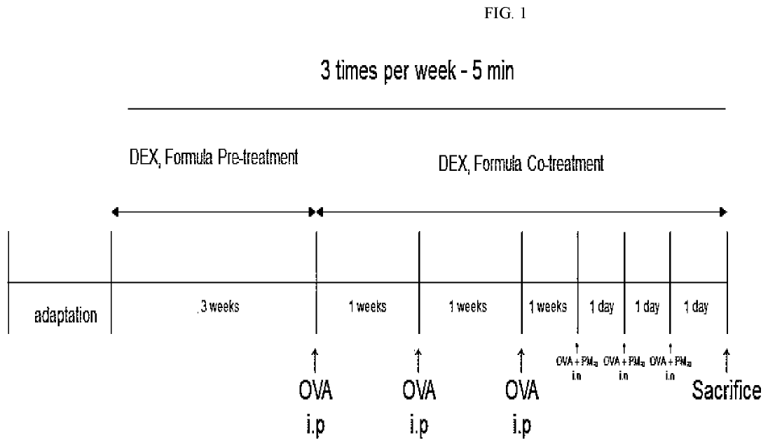

In the mice in the DEX group as the positive control group and in the Low and High groups treated with low or high concentrations of the composite (Formula), samples (DEX or composite) were nebulized for 5 minutes three times a week for 3 weeks in an exposure chamber with a self-made sprayer with a nebulizer (Philips, Amsterdam, Netherlands). The exposure chamber was sealed in a plastic round container by cutting the end of a 50 mL conical tube to 1 cm. The container was connected to the sprayer and then the mice were loaded into a conical tube for exposure to the vapor. The mice in the OVA+PM10 group as the negative control group were nebulized with saline. Thereafter, even for 4 weeks of sensitization with OVA and PM10, the samples were continuously nebulized into the mice with a nebulizer for 5 minutes three times a week. The spray rate of the nebulizer was 1 mL/min. Each sample was pre-treated for 3 weeks, and administered with 10 g of OVA emulsified in 500 g of aluminum hydroxide together with a total volume of 0.1 mL of saline through intraperitoneal injection (i.p.) on days 0, 7, and 15. After sensitization, the mice were challenged with 1 mg of OVA and 100 μg of PM10 supplemented in 50 μL of saline by intranasal injection (i.n.) on days 21 and 22. The DEX group as the positive control group was treated with DEX at a concentration of 2 mg/kg (calculated as 0.06% in saline) and in the case of a high-concentration composite (Formula) treated group (High), 0.4 μL of mint oil, 0.2 μL of Asarum sieboldi oil, and 0.3 μL of fir leaves oil per 1 mL of physiological saline were included to be calculated in a total of 0.09%. The low-concentration treated group was diluted 1/100 and treated at a total concentration of 0.0009%. Thereafter, the mice were sacrificed after 24 days of OVA treatment. The detailed experimental schedule content was illustrated in FIG. 1.

All experimental results using the mice treated with the method were expressed as mean±standard error, an analysis of variance (ANOVA) was calculated from the results of each experiment, and then the significance between the means of each group was tested using a Tukey's multiple range test. In general, a P value of 0.05 or less was considered statistically significant.

### Example 2. Efficacy in Reducing Epithelial Thickness and Collagen Accumulation in Lung Tissue Through Nebulization of Composite

To analyze the efficacy of reducing epithelial thickness and inhibiting collagen accumulation in lung tissue through nebulization of the composite (Formula) in mice with chronic respiratory disease induced by OVA and PM10 using the method of Preparation Example 1, Histological evaluation (Histology) was performed.

Specifically, in order to produce a lung tissue specimen, the lung tissue was fixed in 10% neutral formalin for 24 hours, and then the fixative that had penetrated into the tissue was sufficiently removed through a sufficient washing process. Thereafter, the moisture in the tissue was removed through a dehydration process sequentially with alcohol concentrations of 70%, 90%, 95%, and 100%, and then a paraffin block was produced using xylene as a transparent agent. The completed paraffin block was sectioned by microtome cutting at 5 μm intervals, subjected to deparaffinization and hydration, and then stained with a hematoxylin & eosin (H&E) solution and a Masson's trichrome staining solution. The stained slides were observed under a optical microscope at 400× magnification, and analyzed using the Image J program to analyze the epithelial thickness and the degree of collagen deposition in lung tissue.

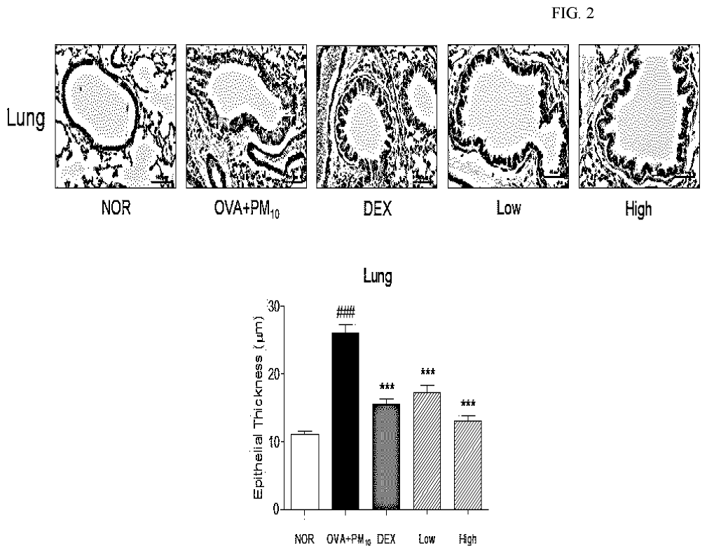

As a result analyzed using H&E staining to evaluate the efficacy of reducing epithelial thickness in lung tissue through nebulization of the composite, it was confirmed that in the group in which mice with chronic respiratory disease induced by OVA and PM10 were treated with a composite (Formula) containing essential oils of mint, Asarum sieboldi, and fir leaves, the epithelial thickness was reduced compared to the negative control OVA+PM10 group treated with saline (FIG. 2).

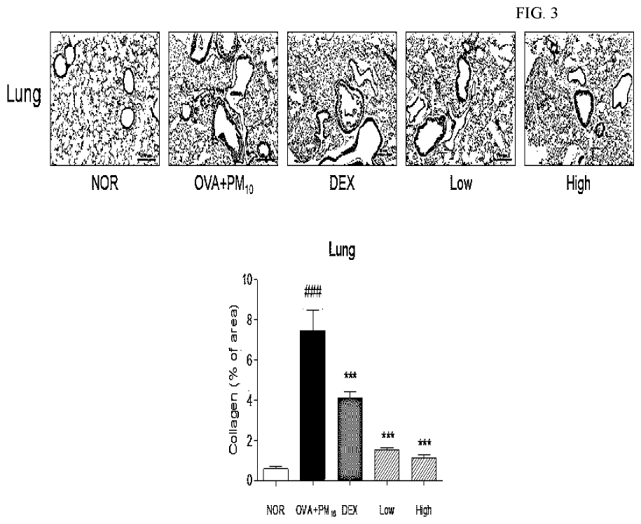

As a result analyzed using Masson's trichrome staining to evaluate the efficacy of inhibiting collagen accumulation in lung tissue through nebulization of the composite, it was confirmed that in the group in which mice with chronic respiratory disease induced by OVA and PM10 were treated with the composite (Formula) containing essential oils of mint, Asarum sieboldi, and fir leaves, the collagen accumulation stained in blue was reduced compared to the negative control OVA+PM10 group treated with saline (FIG. 3).

### Example 3. Efficacy of Reducing Expression Level of IgA in Trachea Tissue Through Nebulization of Composite

In order to produce a trachea tissue specimen, the trachea tissue was fixed in 10% neutral formalin for 24 hours, and then the fixative that had penetrated into the tissue was sufficiently removed through a sufficient washing process. Thereafter, the moisture in the tissue was removed through a dehydration process sequentially with alcohol concentrations of 70%, 90%, 95%, and 100%, and then a paraffin block was produced using xylene as a transparent agent. The completed paraffin block was sectioned by microtome cutting at 5 μm intervals, subjected to deparaffinization and hydration, and then treated with an immunoglobulin A (IgA) primary antibody overnight at 4° C. A biotinylated secondary antibody was reacted for 2 hours and then Avidin-Biotinylated HRP was reacted. IgA was stained with 3-amino-9-ethylcarbazole (AEC) staining and observed under an optical microscope at 400× magnification.

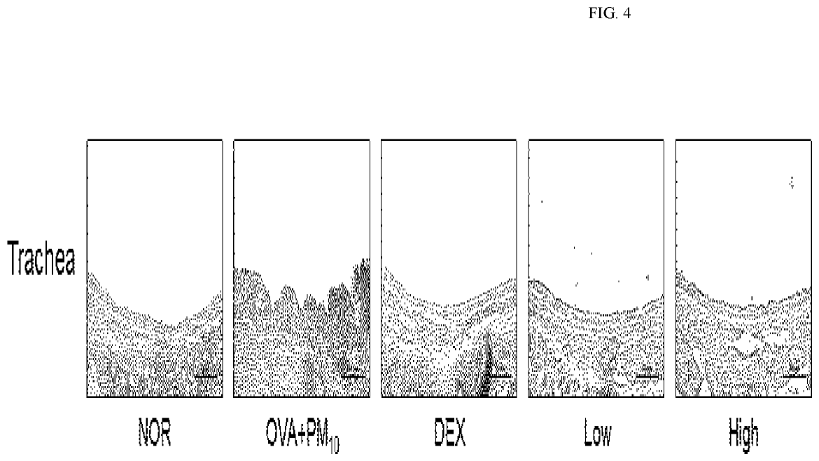

As a result, it was confirmed that in the group in which mice with chronic respiratory disease induced by OVA and PM10 were treated with the composite (Formula) containing essential oils of mint, Asarum sieboldi, and fir leaves, the expression of IgA stained in red was significantly reduced compared to the negative control OVA+PM10 group treated with saline (FIG. 4).

### Example 4. Efficacy in Reducing IgE and IgG Concentrations in Serum Through Nebulization of Composite

To analyze immunoglobulins E and G (IgE and IgG) in serum, heart blood was collected at the end day of the experiment, and the serum was separated by centrifugation (4° C., 15,000 rpm) for 30 minutes. The IgE and IgG levels in serum were measured using an enzyme-linked immunosorbent assays (ELISA) kit according to the manufacturer's instructions. After the measurement reaction was completed, the IgE and IgG concentrations were analyzed at a wavelength of 450 nm using an ELISA reader (Molecular Devices, Downingtown, PA) and calculated using a linear regression equation obtained from standard absorbance values.

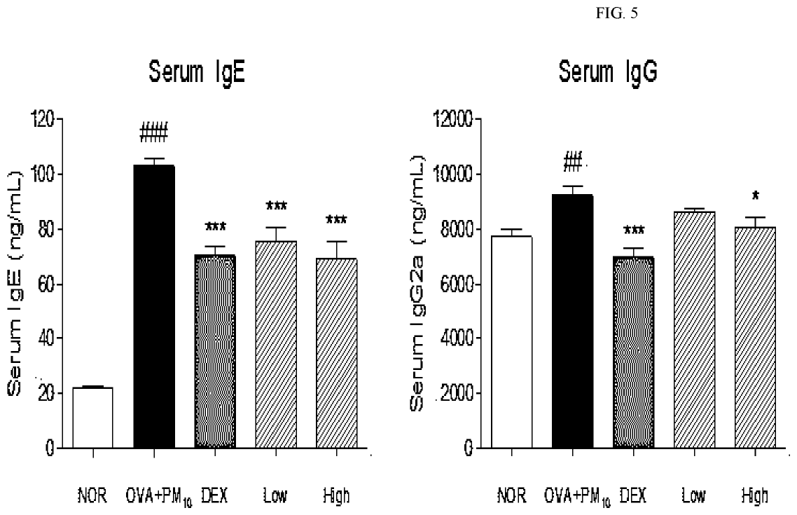

As a result, it was confirmed that in the group in which mice with chronic respiratory disease induced by OVA and PM10 were treated with the composite (Formula) containing essential oils of mint, Asarum sieboldi, and fir leaves, the IgE and IgG concentrations in serum were significantly reduced compared to the negative control OVA+PM10 group treated with saline (FIG. 5).

### Example 5. Efficacy of Inhibiting Inflammatory Cytokines in Lung Tissue Through Nebulization of Composite

RNA was extracted from mouse lung tissue and reverse transcription polymerase chain reaction (RT-PCR) was performed to measure the expression levels of inflammatory cytokines TNF-α and IL-6 in lung tissue.

A total of 1 μg of RNA was sampled according to the instructions of the manufacturer of cDNA synthesis kits (Invitrogen Corp., Carlsbad, CA, USA), and complementary DNA (cDNA) was synthesized through a process for 60 minutes at 45° C. and for 5 minutes at 90° C. The Maxime PCR PreMix Kit (iNtRON, biotechnology, Korea) was used to amplify the inflammatory cytokines TNF-α and IL-6 and a loading control GAPDH from the synthesized cDNA. In a PCR PreMix Kit tube, 2 μl of cDNA, 2 μl of 5 primer and 2 μl of 3′ primer, and 14 μl of distilled water were mixed and amplified in a thermal cycler (Perkin Elmer 2400, USA). The amplified cDNA was electrophoresed on a 1% agarose gel and stained with ethidium bromide to confirm bands. The expression level of each mRNA was measured by Image J (NIH, Bethesda, USA) and quantified using GAPDH as a loading control.

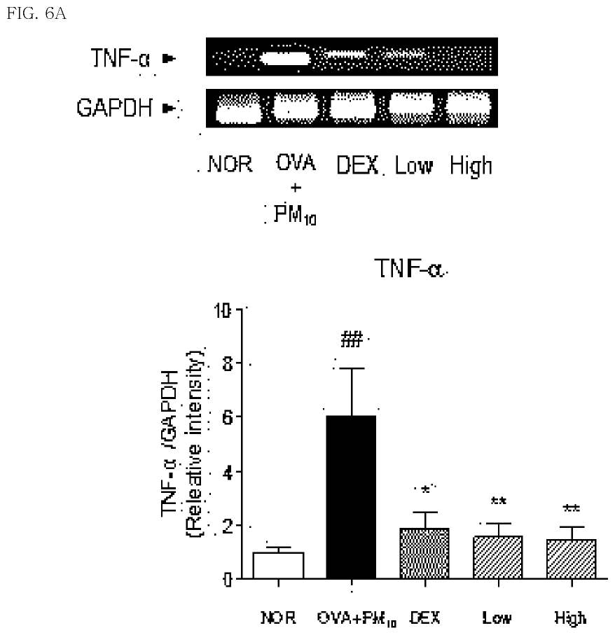

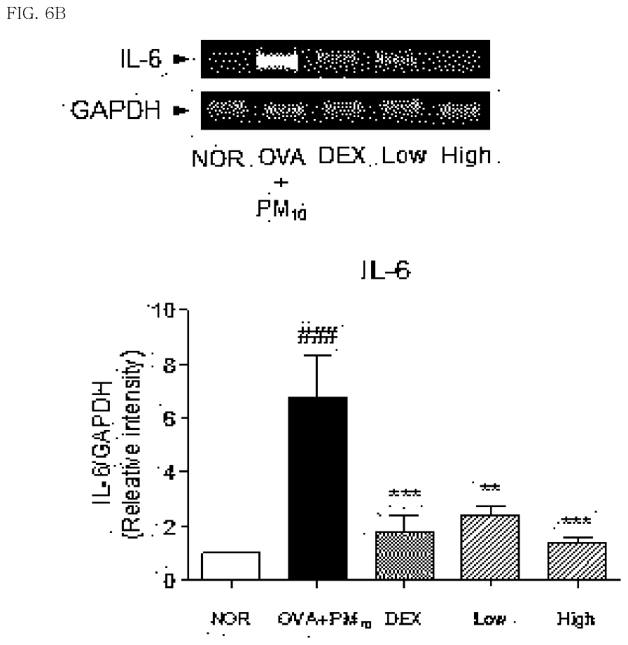

As a result, it was confirmed that in the group in which mice with chronic respiratory disease induced by OVA and PM10 were treated with the composite (Formula) containing essential oils of mint, Asarum sieboldi, and fir leaves, the inflammatory cytokines TNF-α (FIG. 6A) and IL-6 (FIG. 6B) in lung tissue were significantly reduced compared to the negative control OVA+PM10 group treated with saline (FIGS. 6A and 6B).

The present invention has been described above with reference to preferred embodiments thereof.

It will be understood to those skilled in the art that the present invention may be implemented as modified forms without departing from an essential characteristic of the present invention. Therefore, the disclosed embodiments should be considered in an illustrative viewpoint rather than a restrictive viewpoint. The scope of the present invention is illustrated by the appended claims rather than by the foregoing description, and all differences within the scope of equivalents thereof should be construed as being included in the present invention.

1. A method for treating respiratory diseases comprising administering a pharmaceutical composition comprising extracts of mint (Mentha arvensis), Asarum sieboldi (Asiasari Radix et Rhizoma), and fir (Abies holophylla) leaves as active ingredients to a subject in need thereof.

2. The method of claim 1, wherein the extracts of mint, Asarum sieboldi, and fir leaves are essential oil extracts of mint, Asarum sieboldi, and fir leaves, respectively.

3. The method of claim 1, wherein the extracts of mint, Asarum sieboldi, and fir leaves are extracted by a hydrodistillation extraction method.

4. The method of claim 1, wherein the extracts of mint, Asarum sieboldi, and fir leaves are a composite obtained by mixing a mint extract, an Asarum sieboldi extract, and a fir leaves extract in a volume ratio of 4:1 to 3:2 to 4.

5. The method of claim 4, wherein the extracts of mint, Asarum sieboldi, and fir leaves are a composite obtained by mixing the mint extract, the Asarum sieboldi extract, and the fir leaves extract in a volume ratio of 4.0:1.5 to 2.5:2.5 to 3.5.

6. The method of claim 1, wherein the respiratory diseases are respiratory diseases due to particulate matter.

7. The method of claim 6, wherein the respiratory diseases due to the particulate matter are any one selected from the group consisting of asthma, pneumonia, chronic obstructive pulmonary disease, rhinitis, bronchiectasis, acute and chronic bronchitis, bronchiolitis, pharyngitis, tonsillitis, laryngitis, idiopathic pulmonary fibrosis, cystic fibrosis, emphysema, pneumoconiosis, tuberculosis, sequelae of pulmonary tuberculosis, pulmonary fibrosis, lung cancer, lower respiratory infection, sinusitis, acute upper respiratory infection, and allergic lung disease.

8. The method of claim 1, wherein the subject requires reducing increased epithelial thickness and collagen accumulation in lung tissue.

9. The method of claim 1, wherein the subject requires reducing expression or concentration of any one or more of immunoglobulins A, E and G in lung tissue or trachea tissue.

10. The method of claim 1, wherein the subject requires reducing expression of inflammatory cytokines in lung tissue.

11. The method of claim 1, wherein the composition is administered by inhalation or intranasal administration.

12.-13. (canceled)

14. A method treating respiratory diseases comprising administering an inhaled preparation comprising extracts of mint (Mentha arvensis), Asarum sieboldi (Asiasari Radix et Rhizoma), and fir (Abies holophylla) leaves as active ingredients to a subject in need thereof.

15. (canceled)
